(19) United States
(12) Patent Application Publication
(10) Pub. No.: US 2006/0063034 A1
(43) Pub. Date: Mar. 23, 2006

(54) Organic electroluminescent material and organic electroluminescent device by using the same

(75) Inventors: Hung-Lin Chen, Pingtung City (TW); Hsien-Chang Lin, Jubei City (TW); Chia-Dung Wu, Miaoli County (TW); Tsing-Hai Wang, Taipei City (TW)

Correspondence Address: BIRCH STEWART KOLASCH & BIRCH, PO BOX 747, FALLS CHURCH, VA, 22040-0747, US

(73) Assignee: RITDISPLAY CORPORATION

(21) Appl. No.: 11/168,390

(22) Filed: Jun. 29, 2005

(30) Foreign Application Priority Data
Sep. 22, 2004 (TW) 093128778

(51) Int. Cl. H01L 51/54; H05B 33/14; C07D 311/02; C09K 11/06

(52) U.S. Cl. 428/690; 428/917; 313/504; 313/506; 549/280; 549/283

(57) ABSTRACT

An organic electroluminescent material of the formula (1): 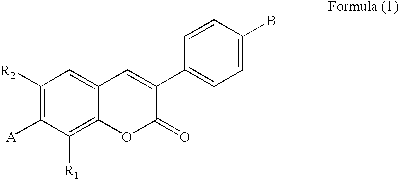
wherein A and B are an electron donating group, R₁ and R₂ are an alkyl group.

### BACKGROUND OF THE INVENTION

1. Field of Invention

The invention relates to an electroluminescent material and an electroluminescent device and, in particular, to an organic electroluminescent material and an organic electroluminescent device.

2. Related Art

The organic electroluminescent device is self-emissive and has excellent properties such as wide viewing angle, high power efficiency, easily to be manufactured, low cost, fast response rate and full color. Therefore, organic electroluminescent device could potentially be the major flat panel display and light source, including being used as special light sources and for normal illumination, in the future.

As mentioned above, the organic electroluminescent device includes a substrate, a first electrode, an organic functional layer and a second electrode. When applying a direct current to the organic electroluminescent device, holes are injected from the first electrode into the organic functional layer while electrons are injected from the second electrode. Based on the applied voltage, the holes and electrons are moved into the organic functional layer, and are recombined to generate excitons then to emit light to release energy. The organic functional layer may include a hole-injecting layer, a hole-transporting layer, a light-emitting layer, an electron-transporting layer, an electron-injecting layer and their combination. The color of the emitted light from the light-emitting layer varies according to the energy gap between ground state and excited state of the materials in the light-emitting layer. The electron-transporting layer and the electron-injecting layer can be composed of organic materials or inorganic materials, which is, for example and not limited to, lithium fluoride (LiF). One of ordinary skill in the art should know that the organic light-emitting display, which is classified into the small molecule OLED (SM-OLED) and the polymer light-emitting display (PLED) according the molecule weights of the organic electroluminescent materials.

As mentioned above, the materials of the organic functional layer have been studied for a long time. For example, W. Helfrish, Dresmer, Williams, et al. succeeded in emission of blue light using anthracene crystals (J. Chen. Phys., 44, 2902 (1966)). Vincett, Barlow, et al. produced a light emitting device by a vapor deposition method, using a condensed polycyclic aromatic compound (Thin Solid Films, 94, 2902 (1982)). However, only a light emitting device with a low luminance and a luminous efficiency has been obtained.

In 1987, C. W. Tang and S. A. Van Slyke disclosed a dual-layer organic functional layer structure having an organic thin film and a transporting thin film, such as a hole-transporting layer or an electron-transporting layer. The organic functional layer has a feature of different colors corresponding to the energy gap between the ground state and excited state of the material therein. It is reported that the maximum luminance provided is more than 1,000 cd/m² and an efficiency of 1 lm/W (Appl. Phys. Lett., Vol. 51, 913 (1987)). After that, scientists developed another organic functional layer structure having three layers to decrease driving voltage of the diode and to increase the maximum luminescence (Japanese Journal of Applied Physics, 1988, 27, 2 L269-L271, and Journal of Applied Physics, 1989, 9 3610). In this case, the organic functional layer structure has an organic light-emitting layer, a hole-transporting layer, and an electron-transporting layer.

The luminescent material is one of the most important materials in the organic electroluminescent device. The selection of luminescent materials must satisfy the following four conditions. First, the luminescent material must have an excellent fluorescent quantum efficiency and a narrow luminescent peak located within the visible light region. Second, the luminescent material must have good semiconductor characteristics, such as the electrical conductivity for conducting electrons, holes, or both. Third, the luminescent material must be easily formed into a film, so that the film would not contain any pinhole. Fourth, the luminescent material must be heat stable. Thus, the luminescent material may not be degraded under high-temperature evaporation, and the film formation may not easily induce the crystallization phenomenon.

Most solid organic dyes have a problem on concentration quenching, which leads to the widened peak or red shift phenomenon of the fluorescence. Therefore, the organic dyes, as the low concentrative guest, are usually doped in the host having the electron- or hole-transporting characteristic (U.S. Pat. No. 4,769,292). The absorption spectrum of the dye and the emission spectrum of the host must be excellent overlapped, so that the energy can be transported from the host to the guest efficiently.

Regarding to blue organic electroluminescent materials, Idemitsu Kosan Company Limited disclosed derivatives of distyrylarylene (DSA) compounds and has many granted patents such as U.S. Pat. Nos. 5,121,029, 5,126,214, 5,130,603, 5,516,577, 5,536,949, 6,093,864, and WO 02/20459. However, the derivatives of distyrylarylene (DSA) compounds still have several drawbacks such as low luminance and emitting efficiency, high driving voltage, color impurity, and et al. For example, as disclosed in U.S. Pat. No. 5,130,603, N,N′-diphenyl-N,N′-bis-(3-methylphenyl)-[1,1′-biphenyl]-4,4′-diamine (TPD) is used in a hole-transporting layer, and 2,5-bis(2,2-di-p-tolyvinyl)xylene (DTVX) is used in an organic light-emitting layer. When applying 5 volts, the luminance of the organic electroluminescent device having TPD and DTVX is 300 cd/m², and the luminescent wavelength of the device is 486 nm. When applying 7 volts, the maximum luminance of the organic electroluminescent device is 1,000 cd/m². In addition, as disclosed in U.S. Pat. No. 5,536,949, TPD is used in the hole-transporting layer, 4,4′-Bis(2,2-diphenylvinyl)biphenyl (DPVBi) is used in the light-emitting layer which doped with 4,4′-Bis[2-{4-(N,N-diphenylamino)pheny}vinyl]biphenyl (DPAVBi), and tris(8-quinolinato-N1,08)-aluminum (AlQ₃) is used in the electron-transporting layer. In this case, when applying 8 volts, the luminance of the organic electroluminescent device is 400 cd/m², and the luminescent wavelength of the device is 494 nm. In U.S. Pat. No. 6,093,864, the organic electroluminescent device has similar properties as mentioned above. Besides, Kodak also disclosed in a U.S. Pat. No. 5,935,721 that the derivatives of perylene compounds can be used as the blue organic functional material. In this case, the host is 9,10-di(2-naphthyl)anthracene (DNA) doped with tetrakis(t-butyl)perylene (TBPe) so as to form a light-emitting layer, wherein AlQ₃ is used in the electron-transporting layer and NPB is used in the hole-transporting layer. The efficiency of Kodak's organic electroluminescent device is 3.2 cd/A when the current density is 20 mA/cm², and the C.I.E. chromaticity coordinates thereof are (X 0.15, Y 0.23). In addition, there are many researches for the blue organic electroluminescent materials, such as Synth. Met. 2001, 118, 193, Adv. Mater. 2001, 13, 1690, Displays, 2001, 22, 61, and J. Mater. Chem. 2001, 11, 768. The disclosed blue organic electroluminescent materials have, however, unsatisfied efficiency of the organic electroluminescent device. Therefore, it is an important subject of the organic electroluminescent device to solve the problems of low efficiency and degradation.

Regarding to the green organic electroluminescent materials, since the derivatives of coumarin compounds have excellent thermal stability and high quantum efficiency, they have been employed (U.S. Pat. No. 4,736,032, JP Pub. 07-166160, and U.S. Pat. No. 6,020,078). For example, the derivatives of coumarin compounds include 10-(2-benzothiazolul)-1,1,7,7-tetramethyl-2,3,6,7-tetrahydro-1H,5H, 11H-[1]benzo-pyr ano[6,7,8-ij]quinolizin-11-one (C545T).

It is therefore an important subject of the invention to provide an organic electroluminescent material and an organic electroluminescent device, which can overcome the above-mentioned problems.

### SUMMARY OF THE INVENTION

In view of the foregoing, the invention is to provide an organic electroluminescent material and an organic electroluminescent device that can solve the problems of low luminescent efficiency and degradation in vacuum evaporation.

To achieve the above, an organic electroluminescent material of the invention has the structure of the following formula (1): 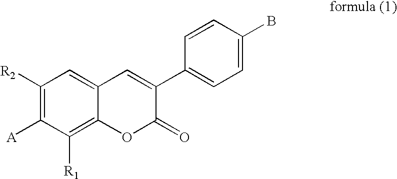
wherein A and B are an electron donating group, R₁ and R₂ are an alkyl group.

To achieve the above, the invention also discloses an organic electroluminescent device, which comprises a substrate, a first electrode, a second electrode and a light-emitting layer. In the invention, the first electrode, the light-emitting layer and the second electrode are disposed over the substrate in sequence. The light-emitting layer comprises an organic electroluminescent material of the following formula (2): 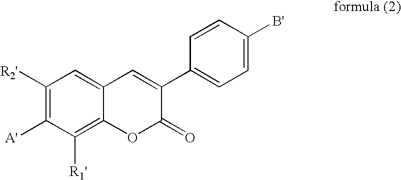
wherein A′ and B′ are an electron donating group, R₁′ and R₂′ are an alkyl group.

As mentioned above, the organic electroluminescent material of the invention has the structure of the formula (1) or the formula (2), which is a derivative of coumarin compounds having excellent thermal stability and high quantum efficiency. In the invention, the disclosed derivative of coumarin compounds includes the electron donating group for adjusting the luminescent color and enhancing luminescent efficiency. In addition, when the organic electroluminescent material of the invention is evaporated at low pressure and high temperature, it would not be degraded easily. That is, the organic electroluminescent material of the invention has higher thermal stability. Thus, the manufacturing difficulty of product can be decreased, and the product stability can be increased. In brief, the organic electroluminescent material and organic electroluminescent device of the invention have not only the increased luminescent efficiency and thermal stability, but also have improved lifetime and manufacturing stability.

### BRIEF DESCRIPTION OF THE DRAWINGS

The invention will become more fully understood from the detailed description given herein below illustration only, and thus is not limitative of the present invention, and wherein:

FIG. 1 is a schematic view showing an organic electroluminescent device according a second preferred embodiment of the invention;

FIG. 2 is a coordinate figure showing the EL spectrum of the organic electroluminescent device according the preferred embodiment of the invention;

FIG. 3 is a coordinate figure showing the I-B curve of the organic electroluminescent device according the preferred embodiment of the invention; and

FIG. 4 is a coordinate figure showing the I-E curve of the organic electroluminescent device according the preferred embodiment of the invention.

### DETAILED DESCRIPTION OF THE INVENTION

The present invention will be apparent from the following detailed description, which proceeds with reference to the accompanying drawings, wherein the same references relate to the same elements.

### First Embodiment

An organic electroluminescent material according to a first preferred embodiment of the invention has the structure of the following formula (1): 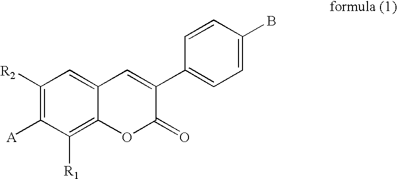
wherein A and B are an electron donating group, R₁ and R₂ are an alkyl group.

In this embodiment, A and B are one selected from the group consisting of a substituted amino group having 1 to 30 carbon atoms, a non-substituted amino group having 1 to 30 carbon atoms, an alkoxy group having 1 to 10 carbon atoms, and an aroxy group having 1 to 30 carbon atoms.

R₁ and R₂ are one selected from the group consisting of a substituted alkyl group having 1 to 6 carbon atoms and a non-substituted alkyl group having 1 to 6 carbon atoms.

In addition, R₁ can bond with A. Alternatively, R₂, of course, can bond with A, too.

For instance, the organic electroluminescent material of this embodiment can be but not limited to one compound of the following formulas:

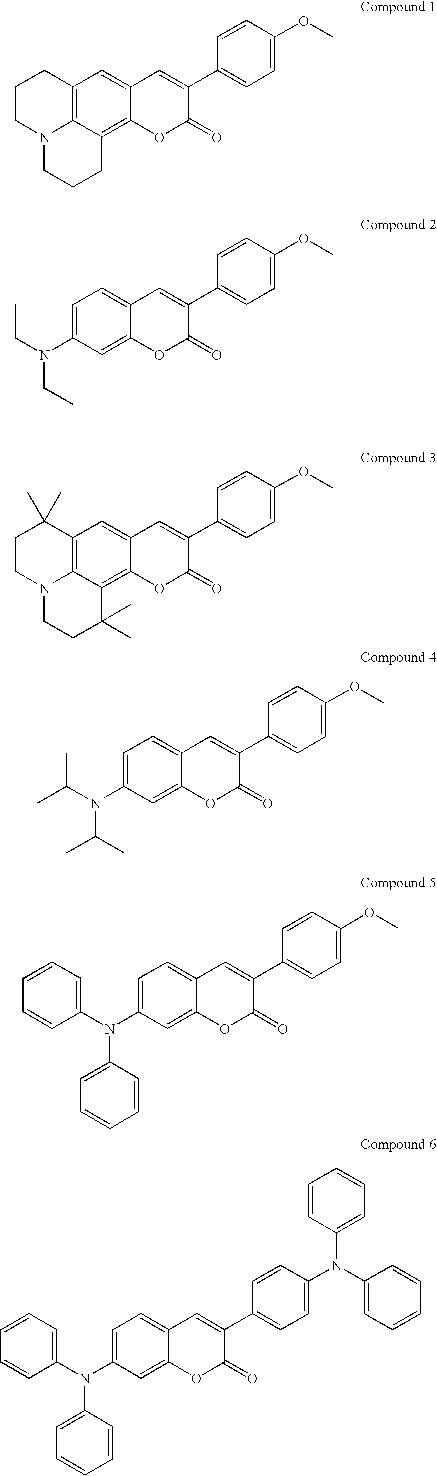

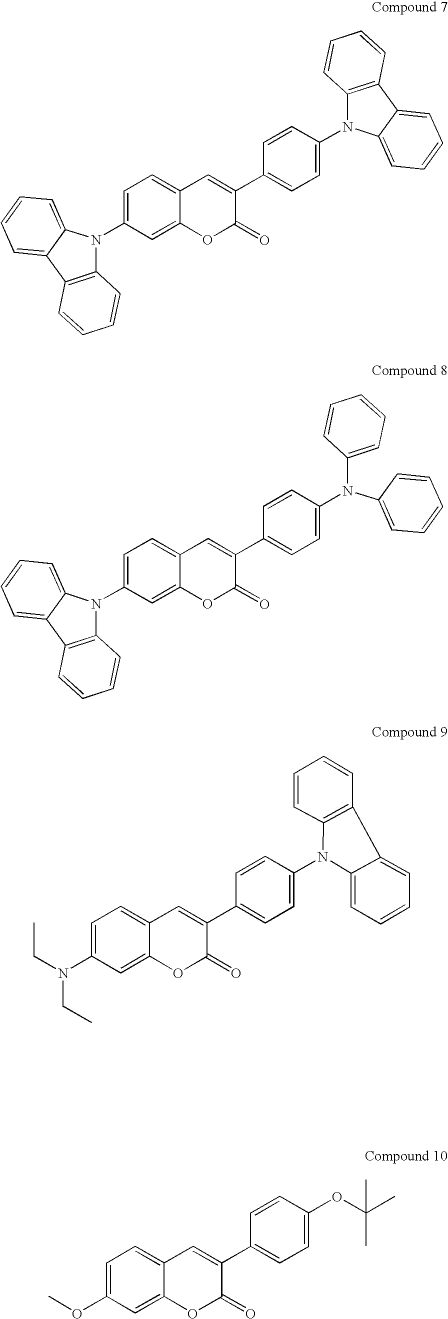

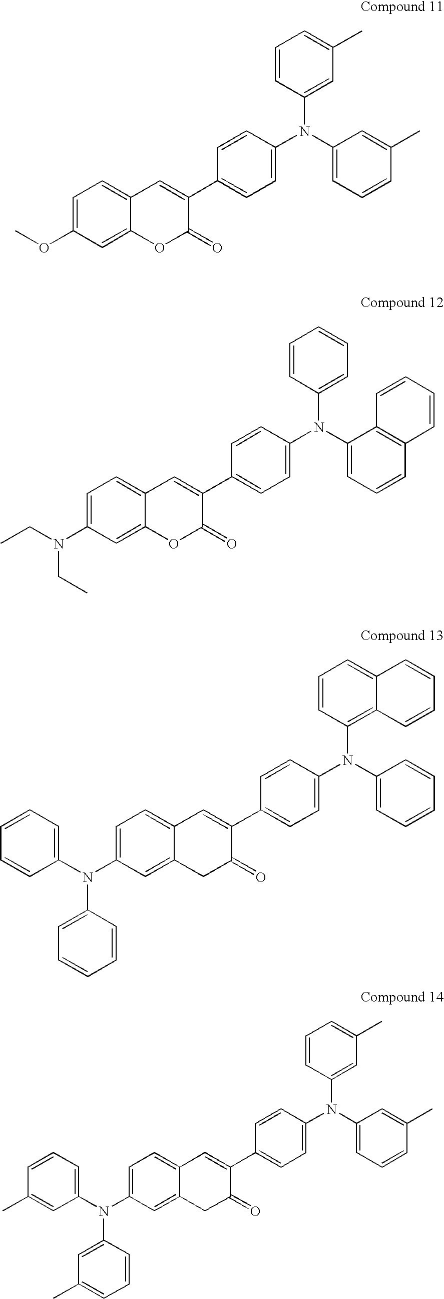

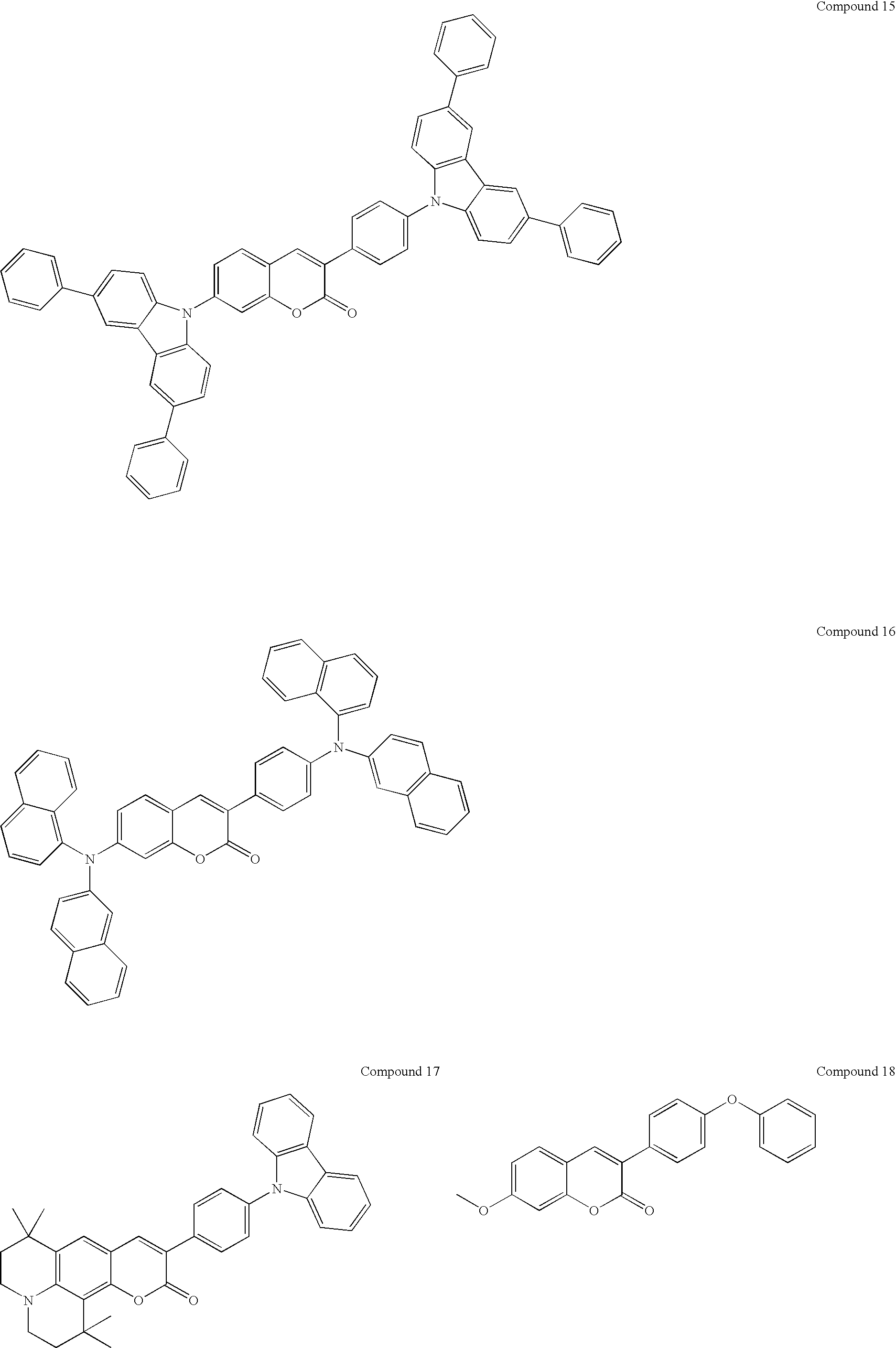

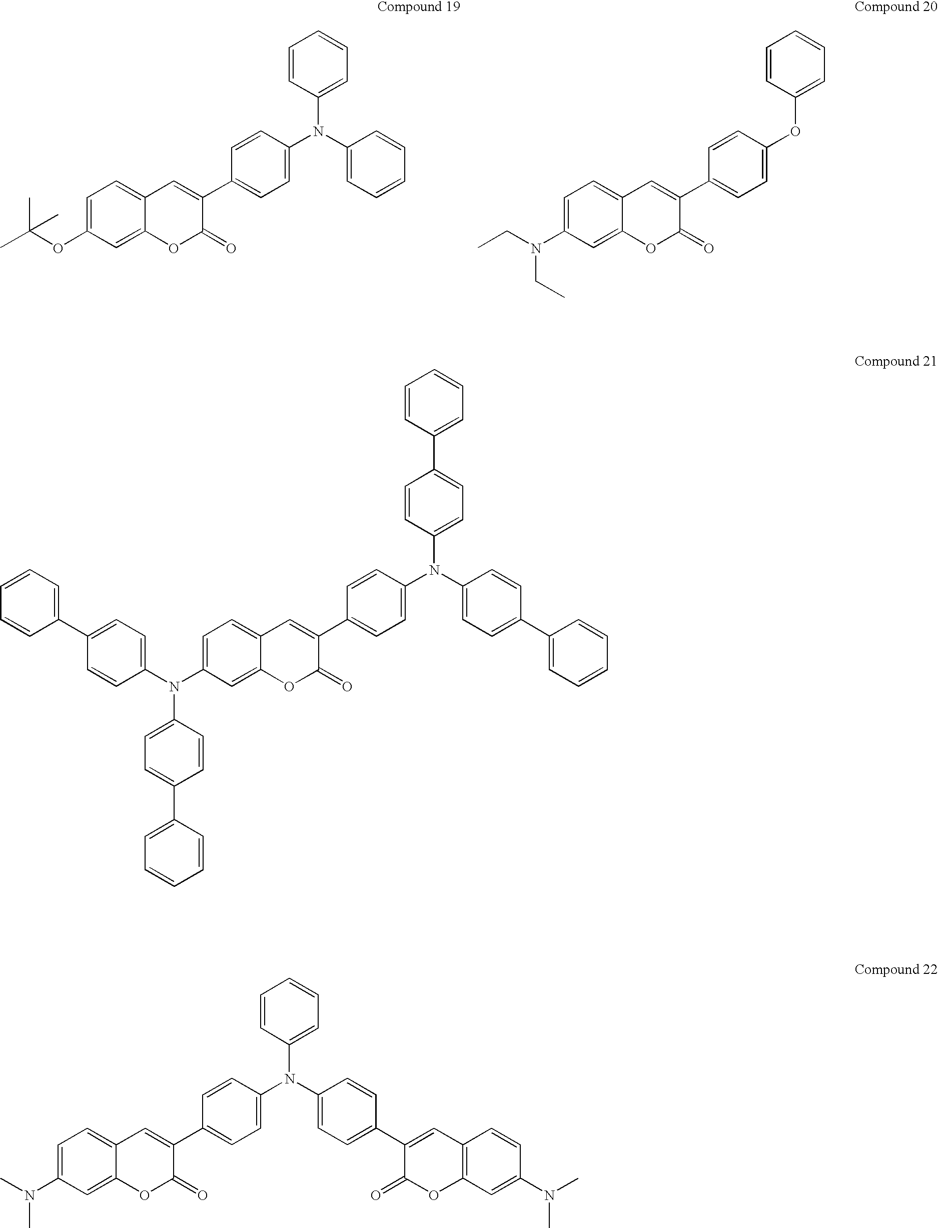

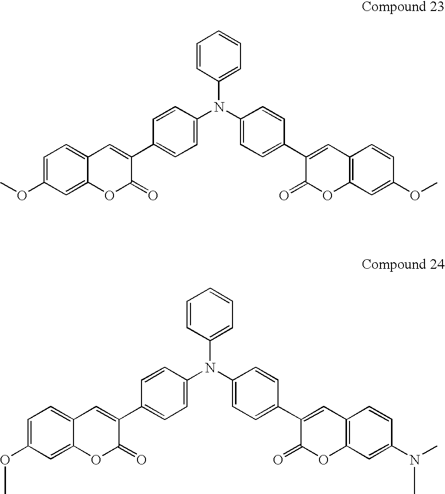

### Second Embodiment

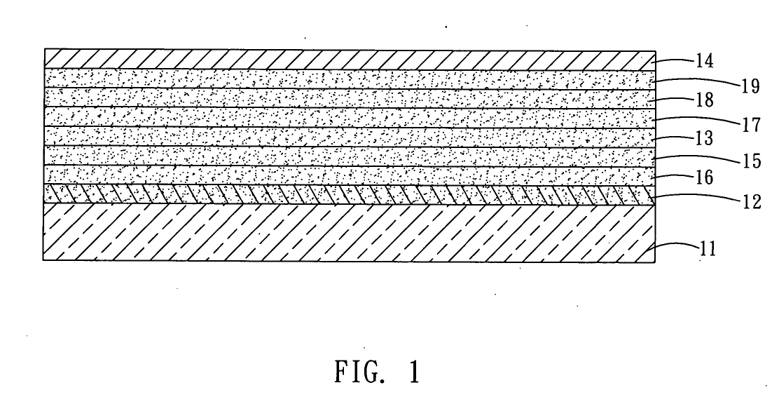

With reference to FIG. 1, an organic electroluminescent device 1 according to a second preferred embodiment of the invention comprises a first electrode 12, a light-emitting layer 13 and a second electrode 14 disposed over the substrate 11 in sequence. The light-emitting layer 13 comprises an organic electroluminescent material of the following formula (2): 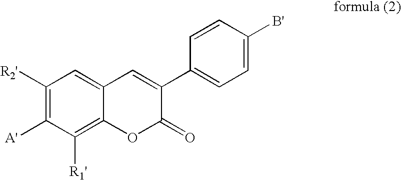
wherein A′ and B′ are an electron donating group, R₁′ and R₂′ are an alkyl group.

In the second embodiment, A′ and B′ are one selected from the group consisting of a substituted amino group having 1 to 30 carbon atoms, a non-substituted amino group having 1 to 30 carbon atoms, an alkoxy group having 1 to 10 carbon atoms, and an aroxy group having 1 to 30 carbon atoms.

R₁′ and R₂′ are one selected from the group consisting of a substituted alkyl group having 1 to 6 carbon atoms and a non-substituted alkyl group having 1 to 6 carbon atoms.

In addition, R₁′ can bond with A′. Alternatively, R₂′, of course, can bond with A′, too.

In the present embodiment, the substrate 11 can be a flexible or a rigid substrate. The substrate 11 can also be a plastic or a glass substrate. In particular, the flexible substrate or plastic substrate comprises polycarbonate (PC), polyester (PET), cyclic olefin copolymer (COC) and metallocene-based cyclic olefin copolymer (mCOC). In addition, the substrate 11 can be a silicon substrate.

The first electrode 12 is formed on the substrate 11 by a sputtering method or an ion plating method. The first electrode 12 is usually used as an anode and made of a transparent conductive metal oxide, such as indium-tin oxide (ITO), aluminum-zinc oxide (AZO), indium-zinc oxide (IZO) or cadmium-tin oxide (CdSnO).

In the current embodiment, the light-emitting layer 13 may be formed upon the first electrode 12 by utilizing evaporation, molecular beam epitaxy (MBE), immersion, spin coating, casting, bar coding, roll coating, printing, ink-jet printing or transferring. Herein, the organic electroluminescent material is ranged from 0.1 wt % to 25 wt % of the light-emitting layer 13.

In addition, the second electrode 14 is disposed over the light-emitting layer 13. Herein, the second electrode 14 can be formed by evaporation or sputtering. The material of the second electrode 14 can be but not limited to aluminum, calcium, magnesium, indium, zinc, manganese, silver, gold and magnesium alloy. The magnesium alloy can be, for example but not limited to, Mg:Ag alloy, Mg:In alloy, Mg:Sn alloy, Mg:Sb alloy or Mg:Te alloy.

Of course, the organic electroluminescent device 1 may further comprise a hole-transporting layer 15 disposed between the first electrode 12 and the light-emitting layer 13. In this case, the hole-transporting layer 15 can be formed by evaporation, spin coating, ink-jet printing, transferring or printing.

Furthermore, the organic electroluminescent device 1 may further comprise a hole-injecting layer 16 disposed between the first electrode 12 and the light-emitting layer 13. In this case, the hole-injecting layer 16 can be formed by evaporation, spin coating, ink-jet printing, transferring or printing.

In this embodiment, the hole-transporting layer 15 and the hole-injecting layer may comprise one of triarylamine group compounds such as, for example but not limited to, the following formulas (H-13) to (H-16).

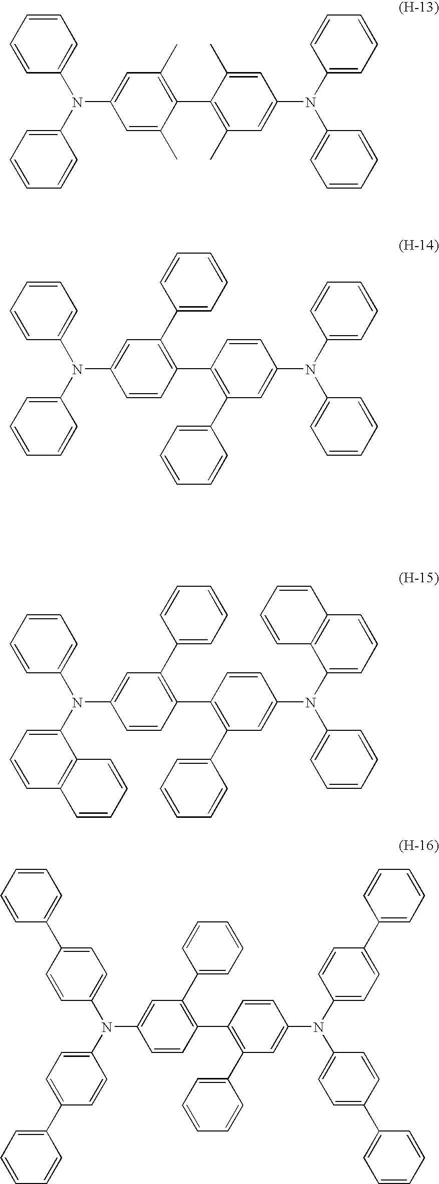

Certainly, the organic electroluminescent device 1 may also comprise a hole-blocking layer 17 disposed between the light-emitting layer 13 and the second electrode 14. The hole-blocking layer 17 functions to block the delivering hole(s) and has a HOMO (Ip) value higher than that of the light-emitting layer 13. Herein, the hole-blocking layer 17 can be formed by evaporation, spin coating, ink-jet printing, transferring or printing.

Of course, the organic electroluminescent device 1 may further comprise an electron-transporting layer 18 disposed between the light-emitting layer 13 and the second electrode 14. Herein, the electron-transporting layer 18 can be formed by evaporation, spin coating, ink-jet printing, transferring or printing.

Herein, the common-used material of the hole-blocking layer 17 and electron-transporting layer 18 can be but not limited to one compound of the following formulas (E-1) to (E-7):

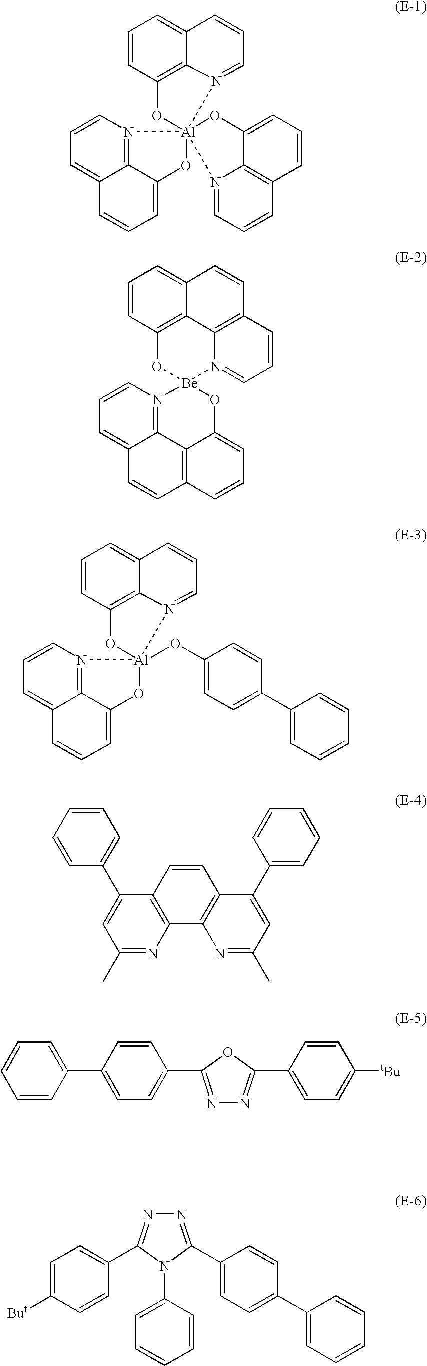

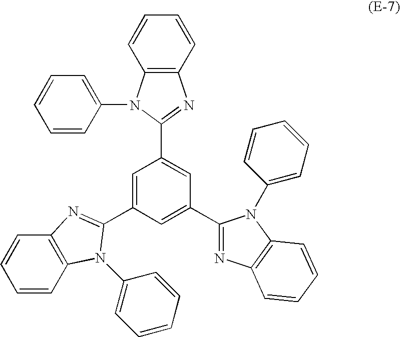

Furthermore, the organic electroluminescent device 1 may further comprise an electron-injecting layer 19 disposed between the light-emitting layer 13 and the second electrode 14.

The above-mentioned hole-injecting layer 16, hole-transporting layer 15, light-emitting layer 13, hole-blocking layer 17, electron-transporting layer 18 and electron-injecting layer 19 are integrally named an organic functional layer.

As mentioned above, the organic functional layer usually comprises one or more stacked structures, and herein below are examples of the structures of the organic functional layer between the first electrode and the second electrode:
(1) first electrode/hole-transporting layer/light-emitting layer/electron-transporting layer/second electrode;
(2) first electrode/hole-transporting layer/light-emitting layer/electron-transporting layer/electron-injecting layer/second electrode;
(3) first electrode/hole-transporting layer/light-emitting layer/hole-blocking layer/electron-transporting layer/electron-injecting layer/second electrode;
(4) first electrode/hole-injecting layer/hole-transporting layer/light-emitting layer/hole-blocking layer/electron-transporting layer/electron-injecting layer/second electrode;
(5) first electrode/hole-injecting layer/hole-transporting layer/light-emitting layer/electron-transporting layer/electron-injecting layer/second electrode;
(6) first electrode/hole-injecting layer/light-emitting layer/electron-transporting layer/electron-injecting layer/second electrode;
(7) first electrode/hole-injecting layer/light-emitting layer/hole-blocking layer/electron-transporting layer/electron-injecting layer/second electrode;
(8) first electrode/light-emitting layer/electron-transporting layer/electron-injecting layer/second electrode; and
(9) first electrode/light-emitting layer/hole-blocking layer/electron-transporting layer/electron-injecting layer/second electrode.

To make the above-mentioned embodiments more comprehensive, several examples are illustrated hereinafter for describing the synthesis of the organic electroluminescent material and the manufacturing processes of the organic electroluminescent device.

### EXAMPLE 1

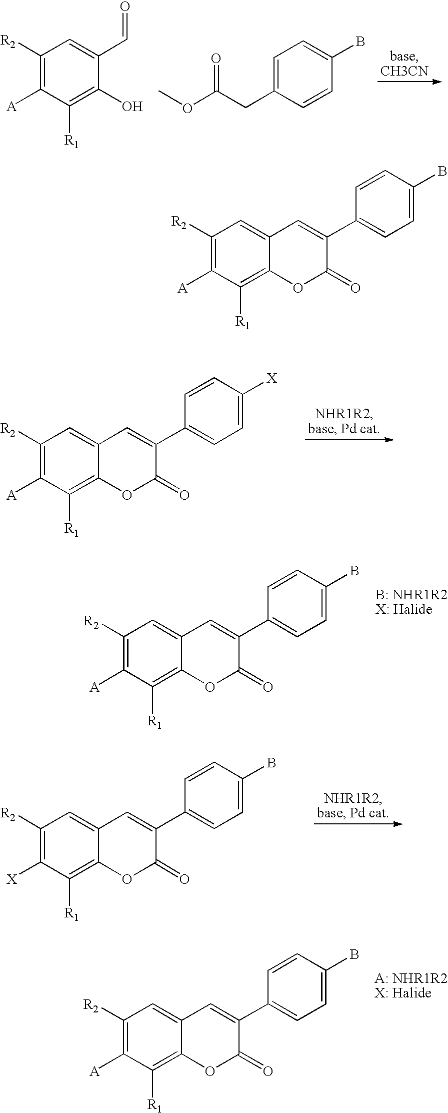

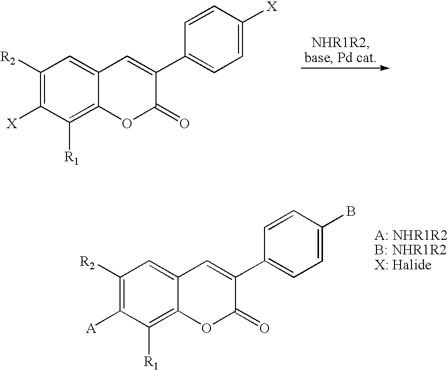

This example illustrates a synthesis method of the organic electroluminescent material according to the embodiment of the invention.

### Compound 2: 7-Diethylamino-3-(4-methoxy-phenyl)-chromen-2-one

First, 4-Diethylamino-2-hydroxy-benzaldehyde (2.000 g, 10.35 mmol), (4-Methoxy-phenyl)-acetic acid methyl ester (1.864 g, 10.35 mmol), and Piperidine (0.881 g, 10.35 mmol) are added into a two-neck bottle. One neck of the two-neck bottle is injected with hydrogen, and the other neck thereof is sealed with a serum plug. After that, 20.0 ml acetonitrile is added, and the solution is then stirred, heated and refluxed for 24 hours. After the temperature of the solution is recovered, ice water is then added to quench the reaction. The solution is extracted by ethyl acetate for several times. The organic layer of the extracted solution is collected and dehydrated by MgSO₄, and then purified by the silica gel column chromatography (mobile phase: EA/n-Hexane=0.2) to obtain a yellow solid compound (0.531 g, 17%). The results of a ¹HNMR (CDCl₃, 400 MHz) analysis of the yellow solid compound (mp. 111.8° C.) are δ 1.20 (t, J=7.2 Hz, 6H), 3.41 (q, J=7.2 Hz, 4H), 3.80 (s, 3H), 6.50 (d, J=2.0 Hz, 1H), 6.58 (dd, J=6.8, 2.0 Hz, 1H), 7.29 (d, J=8.8 Hz, 1H), 7.50 (d, J=8.8 Hz, 2H), 7.56 (d, J=8.0 Hz, 2H), 7.67 (s, 1 Hz).

### EXAMPLE 2

### Compound 11: 3-[4-(Di-m-tolyl-amino)-phenyl]-7-methoxy-chromen-2-one

3-(4-Bromo-phenyl)-7-methoxy-chromen-2-one (2.000 g, 6.04 mmol), Di-m-tolyl-amine (1.251 g, 9.67 mmol), t-BuOK(0.847 g, 7.55 mmol), t-Bu₃P (0.049 g, 0.242 mmol) and Pd₂ dba₃ (0.053 g, 0.060 mmol) are added into a two-neck bottle. One neck of the two-neck bottle is injected with hydrogen, and the other neck thereof is sealed with a serum plug. After that, 12.0 ml toluene is added, and the solution is then stirred, heated and refluxed for 20 hours. Methanol is then added to quench the reaction. The solution is filtered to obtain a yellow solid crude product. After purified by the silica gel column chromatography (mobile phase: EA/n-Hexane=0.2), a yellow solid compound (1.810 g, 70%) is obtained. The results of a ¹HNMR (CDCl₃, 400 MHz) analysis of the yellow solid compound (mp. 151.3° C.) are δ 2.26 (s, 6H), 3.85 (s, 3H), 6.83˜6.88 (m, 4H), 6.90 (d, J=7.2 Hz, 2H), 6.94 (s, 2H), 7.05 (d, J=6.8 Hz, 2H), 7.14 (t, J=6.8 Hz, 2H), 7.40 (d, J=8.8 Hz, 1H), 7.55 (d, J=8.4 Hz, 2H), 7.71 (s, 1H).

### EXAMPLE 3

### Compound 12: 7-Diethylamino-3-[4-(naphthalen-1-yl-phenyl-amino)-phenyl]-chromen-2-one

3-(4-Bromo-phenyl)-7-diethylamino-chromen-2-one (3.000 g, 8.06 mmol), Naphthalen-1-yl-phenyl-amine (2.121 g, 9.67 mmol), t-BuOK (1.357 g, 12.09 mmol), t-Bu₃P (0.163 g, 0.81 mmol) and Pd₂ dba₃ (0.185 g, 0.20 mmol) are added into a two-neck bottle (25 ml). One neck of the two-neck bottle is injected with hydrogen, and the other neck thereof is sealed with a serum plug. After that, 16.0 ml toluene is added, and the solution is then stirred, heated and refluxed for 3 hours. Methanol is then added to quench the reaction. The solution is filtered to obtain a yellow solid crude product. After purified by the silica gel column chromatography (mobile phase: EA/n-Hexane=0.2), a yellow solid compound (2.910 g, 71%) is obtained. The results of a ¹HNMR (CDCl₃, 400 MHz) analysis of the yellow solid compound (mp. 201.1° C.) are δ 1.20 (t, J=7.2 Hz, 6H), 3.40 (q, J=7.2 Hz, 4H), 6.51 (s, 1H), 6.56 (dd, J=6.4, 2.4 Hz, 1H), 6.93 (t, J=7.2 Hz, 1H), 7.00 (d, J=8.8 Hz, 2H), 7.06 (d, J=8.4 Hz, 2H), 7.19 (t, J=8.8 Hz, 2H), 7.28 (d, J=8.0 Hz, 1H), 7.34 (t, J=8.4 Hz, 2H), 7.44 (t, J=7.6 Hz, 2H), 7.51 (d, J=6.8 Hz, 2H), 7.61 (s, 1H), 7.76 (d, J=8.4 Hz, 1H), 7.86 (d, J=8 Hz, 1H), 7.92 (d, J=8 Hz, 1H).

### EXAMPLE 4

### Compound 14: 7-(Di-m-tolyl-amino)-3-[4-(di-m-tolyl-amino)-phenyl]-chromen-2-one

3-(4-Bromo-phenyl)-7-iodo-chromen-2-one (1.400 g, 3.28 mmol), Di-m-tolyl-amine (1.358 g, 6.88 mmol), t-BuOK (0.920 g, 8.20 mmol), t-Bu₃P (0.027 g, 0.131 mmol) and Pd₂ dba₃ (0.030 g, 0.328 mmol) are added into a two-neck bottle. One neck of the two-neck bottle is injected with hydrogen, and the other neck thereof is sealed with a serum plug. After that, 6.5 ml toluene is added, and the solution is then stirred, heated and refluxed for 4 hours. Methanol is then added to quench the reaction. The solution is filtered to obtain a yellow solid crude product. After purified by the silica gel column chromatography (mobile phase: EA/n-Hexane=0.2), a yellow solid compound (1.180 g, 59%) is obtained. The results of a ¹HNMR (CDCl₃, 400 MHz) analysis of the yellow solid compound (mp. 216.8° C.) are δ 2.25 (s, 6H), 2.29 (s, 6H), 6.83 (t, J=8.0 Hz, 4H), 6.89 (d, J=8.0 Hz, 2H), 6.91˜7.00 (m, 7H), 7.04 (d, J=8.8 Hz, 2H), 7.13 (t, J=8.4 Hz, 2H), 7.16˜7.22 (m, 4H), 7.53 (d, J=8.0 Hz, 2H), 7.67 (s, 1H).

### EXAMPLE 5

This example illustrates the manufacturing of the electroluminescent device according to the embodiment of the invention.

First, a 100 mm×100 mm substrate is provided, wherein an ITO layer with a thickness of 150 nm is formed over the substrate. After photolithography and patterning processes, a pattern of 10 mm×10 mm emitting region is defined. In the condition of 10⁻⁵ Pa, a hole-transporting layer with a thickness of 35 nm is coated, and the evaporation ratio of the hole-transporting material, such as the following NPB (N,N′-diphenyl-N,N′-bis-(1-naphthalenyl)-[1,1′-biphenyl]-4,4′-diamine), is maintained at 0.2 nm/sec.

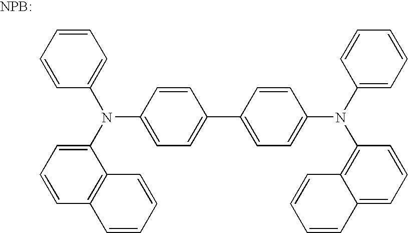

After that, a layer of DNA (9,10-Di-naphthalen-2-yl-anthracene) as shown below and the organic electroluminescent material as the above mentioned compound 7 are co-evaporated to form a light-emitting layer. The weight ratio of DNA and compound 7 in the light-emitting layer is 100:2.5, and the thickness of the light-emitting layer is 45 nm. The evaporation ratio of the materials including DNA and compound 7 is maintained at 0.2 nm/sec.

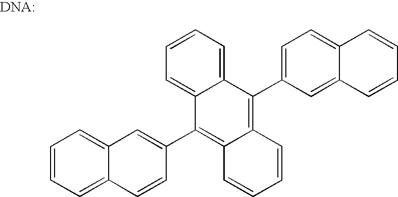

Then, AlQ₃ (tris(8-quinolino)aluminum) of the following structure is coated to form an electron-transporting layer with a thickness of 20 nm. The evaporation ratio of AlQ₃ is maintained at 0.2 nm/sec

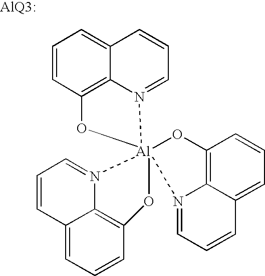

Finally, lithium fluoride (LiF) and aluminum (Al) are formed over the electron-transporting layer as a cathode, and have a thickness of 1.2 nm and 150 nm, respectively. Following the steps, an organic electroluminescent device according to an embodiment of this invention is completed.

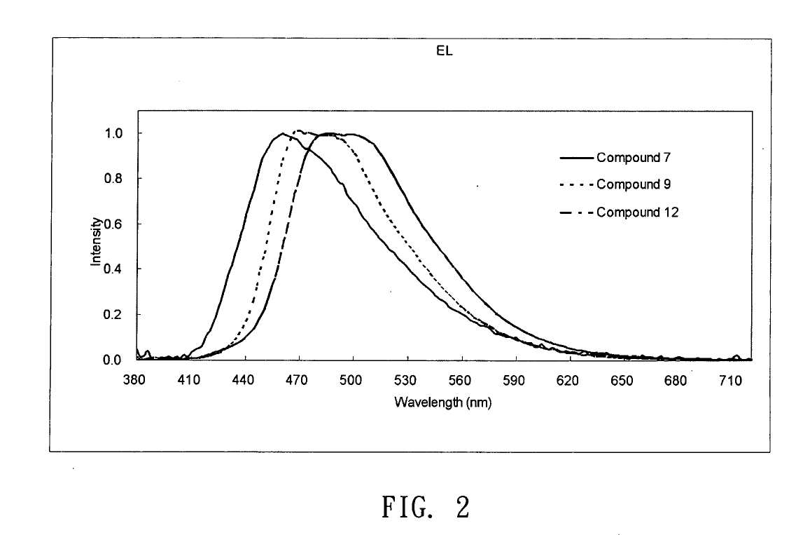

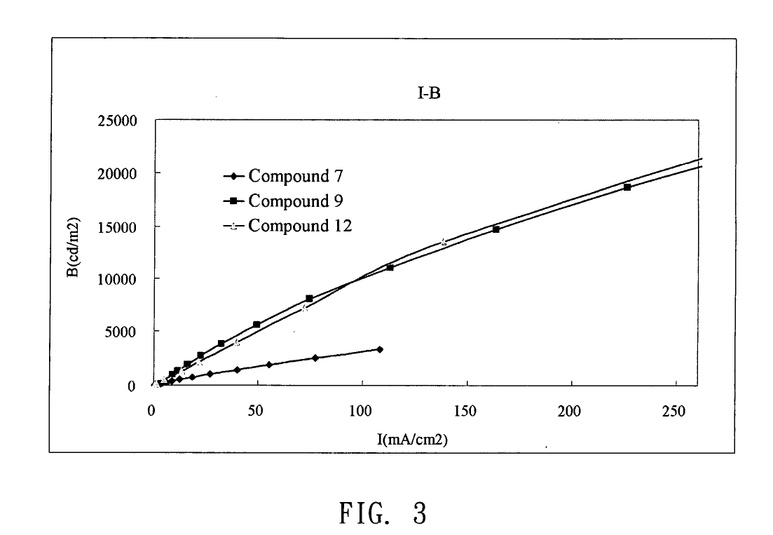

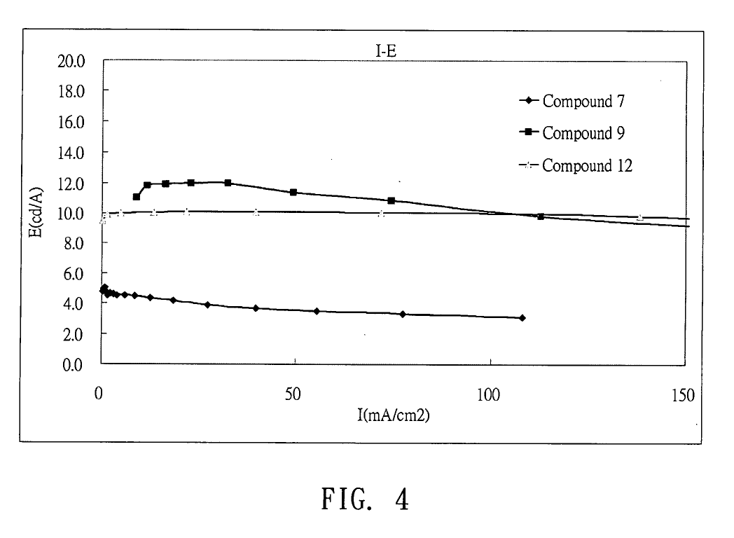

In this case, the luminescent qualities of the organic electroluminescent device according to the embodiment are driven by direct current and are measured by using Keithly 2000. Accordingly, the organic electroluminescent device emits blue light. Furthermore, the EL spectrum of the organic electroluminescent device is measured using a spectrum meter manufactured by Otsuka Electronic Co., wherein the detector is a photodiode array. In this case, the spectrum is shown in FIG. 2, and a luminescent wavelength of 460 nm is obtained. Studying the current vs. luminance (I-B) curve shown in FIG. 3 and the current vs. efficiency (I-E) curve shown in FIG. 4 of the manufactured organic electroluminescent device, we can find that when 12.0 mA/cm² is applied, the luminance of the organic electroluminescent device is 532 cd/m², the efficiency is 4.3 cd/A, and the C.I.E. chromaticity coordinates are (X=0.16, Y=0.23).

### EXAMPLE 6

When the light-emitting layer of the organic electroluminescent device is composed of DNA and compound 9 (100:4), the spectrum diagram of the organic electroluminescent device is shown as FIG. 2, wherein a luminescent wavelength of 472 nm is obtained. Besides, Studying the current vs. luminance (I-B) curve shown in FIG. 3 and the current vs. efficiency (I-E) curve shown in FIG. 4 of the manufactured organic electroluminescent device, we can find that when 11.5 mA/cm² is applied, the luminance of the organic electroluminescent device is 1355 cd/m², the efficiency is 11.8 cd/A, and the C.I.E. chromaticity coordinates are (X=0.17, Y=0.31).

### EXAMPLE 7

When the light-emitting layer of the organic electroluminescent device is composed of DNA and compound 12 (100:2.5), the spectrum diagram of the organic electroluminescent device is shown as FIG. 2, wherein a luminescent wavelength of 496 nm is obtained. Besides, Studying the current vs. luminance (I-B) curve shown in FIG. 3 and the current vs. efficiency (I-E) curve shown in FIG. 4 of the manufactured organic electroluminescent device, we can find that when 13.1 mA/cm² is applied, the luminance of the organic electroluminescent device is 1315 cd/m², the efficiency is 10.0 cd/A, and the C.I.E. chromaticity coordinates are (X=0.19, Y=0.41).

According to the above examples, the organic electroluminescent material of the formula (1) or (2) can be used as the blue organic electroluminescent material. In addition, the organic electroluminescent device manufactured with the organic electroluminescent material of the formula (1) or (2) has excellent luminescent efficiency.

In summary, the organic electroluminescent material of the invention has the structure of the formula (1) or the formula (2), which is a derivative of coumarin compounds having excellent thermal stability and high quantum efficiency. In the invention, the disclosed derivative of coumarin compounds includes the electron donating group for adjusting the luminescent color and enhancing luminescent efficiency. In addition, when the organic electroluminescent material of the invention is evaporated at low pressure and high temperature, it would not be degraded easily. In other words, the organic electroluminescent material of the invention has a higher thermal stability. Thus, the difficulty of product manufacturing can be decreased, and the product stability can be increased. In brief, the organic electroluminescent material and organic electroluminescent device of the invention have not only the increased luminescent efficiency and thermal stability, but also have improved lifetime and manufacturing stability.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. An organic electroluminescent material of the formula (1): 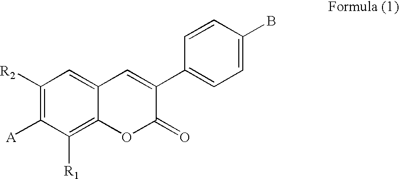
wherein A and B are an electron donating group, R₁ and R₂ are an alkyl group.

2. The organic electroluminescent material of claim 1, wherein A and B are one selected from the group consisting of a substituted amino group having 1 to 30 carbon atoms, a non-substituted amino group having 1 to 30 carbon atoms, an alkoxy group having 1 to 10 carbon atoms, and an aroxy group having 1 to 30 carbon atoms.

3. The organic electroluminescent material of claim 1, wherein R₁ and R₂ are one selected from the group consisting of a substituted alkyl group having 1 to 6 carbon atoms and a non-substituted alkyl group having 1 to 6 carbon atoms.

4. The organic electroluminescent material of claim 1, wherein R₁ bonds with A.

5. The organic electroluminescent material of claim 1, wherein R₂ bonds with A.

6. An organic electroluminescent device, comprising:
a substrate;
a first electrode;
a second electrode; and
a light-emitting layer, wherein the first electrode, the light-emitting layer and the second electrode are disposed over the substrate in sequence, and the light-emitting layer comprises an organic electroluminescent material of the formula (2): 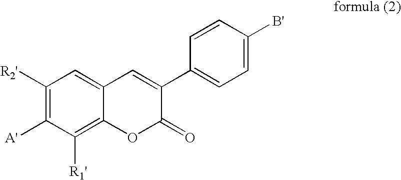
wherein A′ and B′ are an electron donating group, R₁′ and R₂′ are an alkyl group.

7. The organic electroluminescent device of claim 6, wherein A′ and B′ are one selected from the group consisting of a substituted amino group having 1 to 30 carbon atoms, a non-substituted amino group having 1 to 30 carbon atoms, an alkoxy group having 1 to 10 carbon atoms, and an aroxy group having 1 to 30 carbon atoms.

8. The organic electroluminescent device of claim 6, wherein R₁′ and R₂′ are one selected from the group consisting of a substituted alkyl group having 1 to 6 carbon atoms and a non-substituted alkyl group having 1 to 6 carbon atoms.

9. The organic electroluminescent device of claim 6, wherein R₁′ bonds with A′.

10. The organic electroluminescent device of claim 6, wherein R₂‘bonds with A’.

11. The organic electroluminescent device of claim 6, wherein the organic electroluminescent material is ranged from 0.1 wt % to 25 wt % of the light-emitting layer.

12. The organic electroluminescent device of claim 6, further comprising:
a hole-transporting layer disposed between the first electrode and the light-emitting layer.

13. The organic electroluminescent device of claim 6, further comprising:
a hole-injecting layer disposed between the first electrode and the light-emitting layer.

14. The organic electroluminescent device of claim 6, further comprising:
a hole-blocking layer disposed between the light-emitting layer and the second electrode.

15. The organic electroluminescent device of claim 6, further comprising:
an electron-transporting layer disposed between the light-emitting layer and the second electrode.

16. The organic electroluminescent device of claim 6, further comprising:
an electron-injecting layer disposed between the light-emitting layer and the second electrode.

17. The organic electroluminescent device of claim 6, wherein the substrate is at least one selected from the group consisting of a rigid substrate, a flexible substrate, a glass substrate, a plastic substrate and a silicon substrate.

18. The organic electroluminescent device of claim 6, wherein the first electrode comprises conductive metal oxide.

19. The organic electroluminescent device of claim 18, wherein the conductive metal oxide is at least one selected from the group consisting of indium-tin oxide, aluminum-zinc oxide, indium-zinc oxide and cadmium-tin oxide.

20. The organic electroluminescent device of claim 6, wherein the second electrode is made of at least one material selected from the group consisting of aluminum, calcium, magnesium, indium, zinc, manganese, silver, gold and magnesium alloy.

21. The organic electroluminescent device of claim 20, wherein the magnesium alloy comprises at least one selected from the group consisting of Mg:Ag alloy, Mg:In alloy, Mg:Sn alloy, Mg:Sb alloy and Mg:Te alloy.
